(19) United States
(12) Patent Application Publication
(10) Pub. No.: US 2008/0221502 A1
(43) Pub. Date: Sep. 11, 2008

(54) Absorbent Tampon Providing Clean Digital Insertion

(75) Inventors: Curt Binner, Furlong, PA (US); Melinda Cettina, Robbinsville, NJ (US); Gina J. Marcus, Lawrenceville, NJ (US); George Quirk, Lawrenceville, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: Johnson & Johnson Consumer Companies, Inc., Skillman, NJ (US)

(21) Appl. No.: 10/576,530

(22) PCT Filed: Oct. 21, 2004
(86) PCT No.: PCT/US2004/035234

§ 371 Date: Feb. 19, 2008

Related U.S. Application Data
(60) Provisional application No. 60/516,958, filed on Oct. 31, 2003.

(51) Int. Cl. A61F 13/20

(52) U.S. Cl. 604/11; 604/385.18; 604/385.17; 28/119; 28/120; 28/116

(57) ABSTRACT

A tampon has a generally uniform fiber distribution along its length, an insertion end, a withdrawal end, and a longitudinal axis. A finger recess having a depth of at least about 5 mm is formed into the withdrawal end. Nonetheless, a column strength of at least about 10 Newtons (N) can still be achieved. The tampon can be formed by winding an absorbent fibrous web around a winding mandrel; transferring the blank into a press; inserting a forming mandrel into one end of a tampon blank while the tampon blank is positioned in the press; moving a plurality of press jaws toward a central longitudinal press axis to compress the tampon blank and to form a compressed tampon having a finger recess formed into the one end of the tampon; and ejecting the compressed tampon from the press.

### CROSS-REFERENCE TO RELATED APPLICATIONS

This invention is related to the following copending application: U.S. Ser. No. 10/700,743, filed on the same day herewith, entitled “A Hygienic Tampon and an Absorbent Body Used in the Formation of a Tampon” (Att'y Docket, J&J-5085).

### FIELD OF THE INVENTION

The present invention relates to an absorbent article for insertion into the body and a method for making this absorbent article. The absorbent article has a formed void in the withdrawal end such that it accommodates a finger and aids in insertion into the body.

### BACKGROUND OF THE INVENTION

Tampons for use in the body can typically be categorized into two groups, ones inserted into the body cavity by an applicator or those inserted digitally by the user. Both types are well known and have been commercially available for many years.

The applicator for tampon insertion is made up of multiple parts, including an insertion portion and a plunger. After the tampon is inserted, these parts are then disposed of thereby creating waste.

Digitally inserted tampons are usually more compact in their packaging and provide little waste. Some women, however, are reluctant to use a digital tampon, as insertion may be messier than with a tampon inserted with an applicator. Additionally, placement of the tampon within the vaginal canal may be more precise and easier with an applicator.

Voids have been incorporated into the rear or withdrawal end of tampons for various reasons. For example, German Utility Model 201 15 829.9 (Spodeck) discloses the use of a concave curvature on the bottom of the tampon. This allows for a form-fitting engagement of the user's index finger during insertion. Johnson, U.S. Pat. No. 3,863,636, discloses packing a withdrawal string inside a small diameter longitudinally disposed bore. Other patents disclose tampons having a void such that an insertion aid such as a stick can be placed within the void. See, for example, Olson et al., U.S. Pat. No. 3,683,912, Cloots et al., U.S. Pat. No. 3,131,435, and Truman, U.S. Pat. No. 3,983,875.

Leutwyler et al., U.S. Pat. No. 5,813,102, discloses an apparatus for forming a tampon having a finger recess in the withdrawal end of the tampon. The tip of an index finger is thereafter inserted into the finger recess before the tampon is introduced into the body. The withdrawal end is compacted to a lesser extent, making it easier to shape the finger recess and insert the index finger into the recess. The softer nature of the fiber material at the withdrawal end gives the user a more congenial feeling and allows for a more rapid expansion of the withdrawal end, which is assumed to aid in leakage protection.

Child et al., U.S. Pat. No. 6,283,952, discloses a tampon and a method of forming a tampon wherein absorbent material is rolled to form an elongated pledget. Some of the layers of absorbent material are displaced using a conical die. The pledget is then compressed and heat conditioned. The compressed pledget is then pressed, using a pushrod dimensioned to produce an indentation in the pledget, against a head forming heated die. The displacement of material creates a conical depression or indentation. The indentation facilitates flaring, which provides a finger well for the user even if the user does not flare the withdrawal end. Fiber displacement causes the fiber density to be increased along the central core of the pledget, giving the pushrod a firmer area to push against than it would have in a compressed pledget, which was not subjected to this fiber displacement. The preferred dimension of the pushrod used for forming the indentation can range from 0.39 to 0.69 inches in length.

While there are illustrated developments in providing tampons with recesses at their withdrawal end, a tampon is needed that has sufficient column strength for digital insertion and that provides a sufficiently sized, clean and neat void, which forms a finger recess that is large enough to provide a secure fit on a user's finger during insertion. A process is needed to form such a tampon without significant axial displacement of fiber into a domed insertion end. Such axial displacement can lead to an overly dense and damaged insertion end.

### SUMMARY OF THE INVENTION

It has been discovered that a tampon having a unique finger recess that is large enough to provide a secure fit on a user's finger during insertion provides a clean feeling to the user without destroying the column strength of the tampon.

In one aspect of the invention, the tampon includes a fibrous absorbent body having a generally uniform fiber distribution along its length, an insertion end, a withdrawal end, and a longitudinal axis. A finger recess having a depth of at least about 5 mm is formed into the withdrawal end. Nonetheless, a column strength of at least about 10 Newtons (N) can still be achieved.

In another aspect of the invention, the tampon is formed by winding an absorbent fibrous web around a winding mandrel; transferring the blank into a press; inserting a forming mandrel into one end of a tampon blank while the tampon blank is positioned in the press; moving a plurality of press jaws toward a central longitudinal press axis to compress the tampon blank and to form a compressed tampon having a finger recess formed into the one end of the tampon; and ejecting the compressed tampon from the press.

Another aspect of the invention is an apparatus for manufacturing an absorbent tampon with a finger recess located in its withdrawal end. The apparatus includes (1) a plurality of press jaws that are radially moveable toward a central longitudinal press axis corresponding to the longitudinal axis of the tampon and (2) a forming mandrel insertable into the press along the central longitudinal press axis in a location corresponding to the withdrawal end of the tampon. The press jaws are radially moveable while the forming mandrel is inserted into an end of the press corresponding to the withdrawal end of the tampon.

### BRIEF DESCRIPTION OF THE DRAWING

FIG. 1 is a perspective view of a commercially available o.b.® Normal Absorbency Tampon;

FIG. 2 is cross-section of the tampon of FIG. 1, taken along line 2-2;

FIG. 3 is a perspective view of a tampon blank useful to form the tampon of FIG. 1;

FIG. 4 is a perspective view of a tampon made according to the present invention;

FIG. 5a is a cross-section of the tampon of FIG. 4 taken along line 5a-5a;

FIG. 5b is a cross-section of the tampon of FIG. 4 taken along line 5b-5b;

FIG. 6 is a representation of a user placing a tampon of the present invention on her finger to prepare for its insertion;

FIG. 7 is a side elevation of a winding mandrel useful in the present invention (FIG. 7b) and a cross-section of the winding mandrel, taken along line a-a of FIG. 7b (FIG. 7a);

FIG. 8 is an end view of a tampon blank useful in the present invention;

FIG. 9 is a cross-section of a tampon press (similar to that shown in Leutwyler et al., U.S. Pat. No. 5,911,712) in an open position (FIG. 9a) and closed position (FIG. 9b) with a forming mandrel; and

FIG. 10 is a longitudinal cross-section of a compressed tampon and a transfer rod in sequence: before (FIG. 10a), during (FIG. 10b), and after (FIG. 10c) transfer of the compressed tampon.

### DETAILED DESCRIPTION OF THE PREFERRED EMBODIMENTS As used herein the specification and the claims, the term “spiral” and variants thereof relate to winding around a center or pole and gradually receding from or approaching it.

As used herein the specification and the claims, the term “tampon” and variants thereof relate to a device that absorbs bodily fluids when inserted into a mammalian body. Included in this definition are vaginal and nasal tampons.

As used herein the specification and the claims, the term “void” and variants thereof relate to a macroscopic empty space.

As used herein the specification and the claims, the term “compressible” and variants thereof relate to materials that can be compressed to hold a generally compressed form, but they also can expand to a relatively uncompressed state upon exposure to sufficient moisture.

As used in the specification and claims, the term “radially expand” and variations of this term relate to the expansion of generally cylindrical tampons.

The present invention concerns providing a tampon for insertion into the body that protects the user's finger from being soiled by body exudates. It also provides a means for directing the position of the tampon during insertion. In particular, a fibrous tampon, especially for digital insertion, having a generally uniform fiber distribution along the length of the tampon and providing a finger recess extending at least about 5 mm into the withdrawal end of the tampon is disclosed. The tampon has a column strength of at least about 10 N.

Additionally, the present invention provides a method of making an absorbent article having a void formed in the base of the tampon.

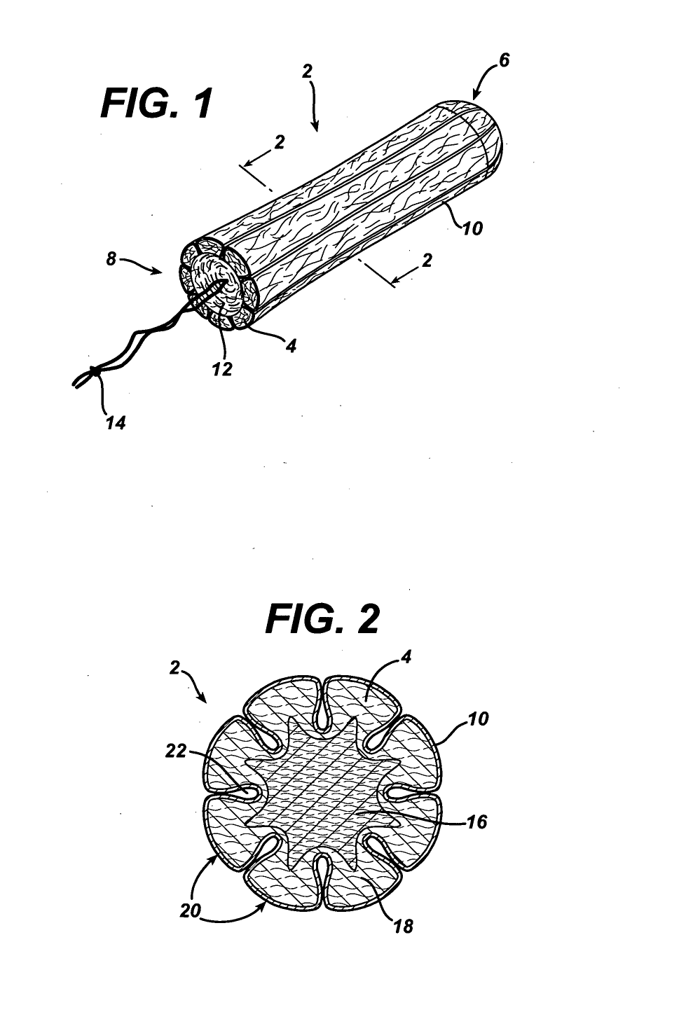

FIG. 1 illustrates a commercially available vaginal tampon 2, e.g., an o.b.® Normal Absorbency Tampon. The tampon 2 has an absorbent structure comprising an absorbent material 4, an introduction end 6, and an opposite withdrawal end 8. An overwrap material 10 is disposed on absorbent material 4 as described, below. The tampon 2 also has a recess 12 in and a withdrawal string 14 extending from the withdrawal end 8. The recess 12 is generally concave and is shown as a series of broken circular lines in FIG. 1, and is generally not more than about 3 mm deep (measured along the product longitudinal axis).

As shown in FIG. 2, the exemplary tampon 2 has a relatively compressed core 16 substantially surrounding the central axis and a less compressed annulus 18 surrounding the core 16. The relatively compressed core 16 may be signaled by increased density in comparison to the annulus 18. This density differential may be provided by relatively uniform fiber distribution within the core 16 and annulus 18, or it may be provided by a plurality of ribs 20 which extend radially from the core 16. In a preferred embodiment, each rib 20 is separated from adjacent ribs where it is attached to the core 16 by an open channel 22, and each rib 20 contacts adjacent ribs proximate the circumferential surface of the tampon.

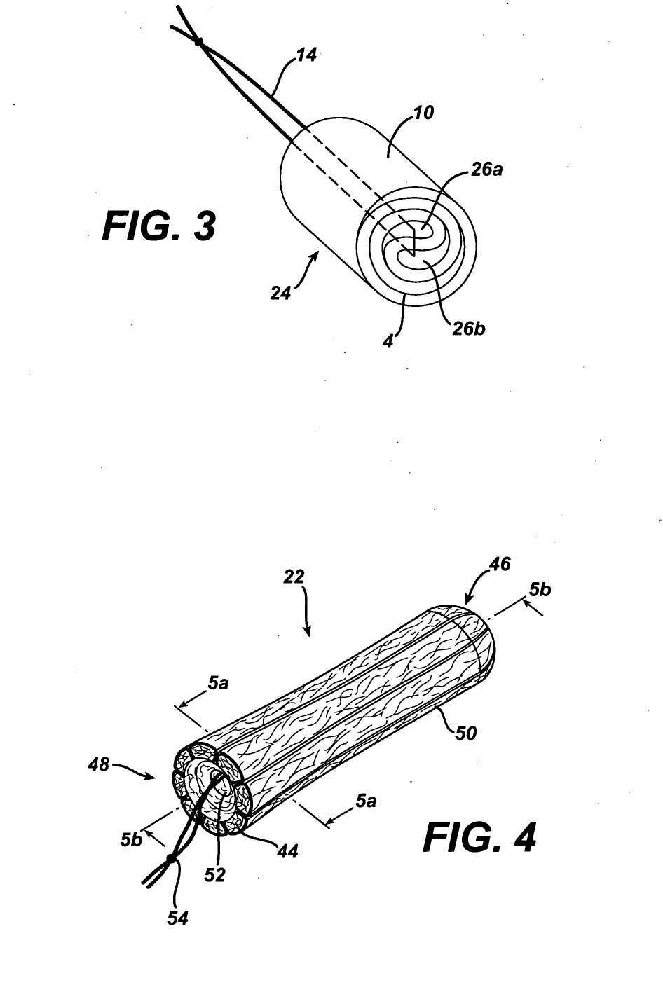

Such commercially available tampons can be made from an apparatus and method such as that disclosed in Friese, U.S. Pat. No. 4,816,100, Friese et al., U.S. Pat. No. 6,310,296, and Leutwyler et al., U.S. Pat. No. 5,911,712. In this method of producing a tampon, an overwrap material is attached to an absorbent web. The absorbent web with the overwrap is then rolled up on itself about an axis extending transversely to its longitudinal direction by a winding mandrel. An example of the rolled structure, or tampon blank 24, is shown in FIG. 3. In this example, the winding mandrel is a fork having two fingers, symmetrically located about the central axis of the mandrel, that are substantially the same size. After rolling, the winding mandrel is removed, leaving two voids shown as 26a and 26b, which are also symmetrical in size and location.

Referring to FIG. 4, there is shown an embodiment of the present invention, a vaginal tampon 42. The tampon 42 has an absorbent structure comprising an absorbent material 44, an introduction end 46, and an opposite withdrawal end 48. The absorbent structure has a generally uniform fiber distribution along the length of the tampon. An overwrap material 50 is disposed on absorbent material 44 in any useful manner, e.g., as described above for the o.b.® Normal Absorbency Tampon. The tampon 42 also has a relatively deep, elongate finger recess 52 in and a withdrawal string 54 extending from the withdrawal end 48.

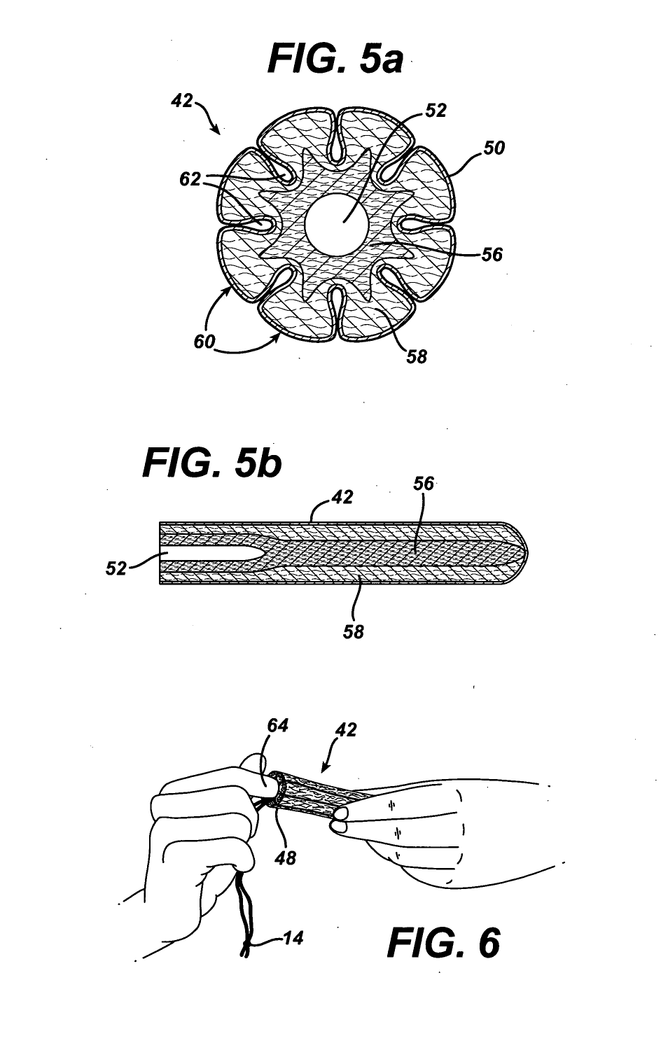

FIG. 5, a cross-section of FIG. 4, shows a relatively compressed core 56 again substantially surrounds the central axis, and a less compressed annulus 58 surrounds the core 56. However, in the region of the withdrawal end 48, the finger recess 52 extends well into the withdrawal end 48 and leaves a void within the core 56. Again, the density differential may be provided by relatively uniform fiber distribution within the core 56 and annulus 58, or it may be provided by a plurality of ribs 60 which extend radially from the core 56. In a preferred embodiment, each rib 60 is separated from adjacent ribs where it is attached to the core 56 by an open channel 62, and each rib 60 contacts adjacent ribs proximate the circumferential surface of the tampon 42.

The recess preferably is of a depth and diameter such that a user can maintain control of the tampon for insertion merely by placing her finger in the finger recess. Preferably, the finger recess has a depth of at least about 5 mm, more preferably at least about 10 mm, and most preferably the recess has a depth of about 10 mm to about 15 mm. These dimensions provide a ratio of depth of finger recess:diameter of finger recess of at least about 1:1 more preferably at least about 2:1, and most preferably the ratio is about 2:1 to about 4:1.

The present invention provides many benefits. In particular, the finger recess 52 allows the user to place the tampon 42 securely on her finger 64 during insertion of the tampon 42 into the body (See FIG. 6). This aids the user in placing the tampon in the vaginal canal and also allows the fingertip to remain clean, i.e., the fingertip portion does not contact the body or bodily fluids during insertion. Potential irritation or scraping from the users fingernail is also minimized. Finger insertion into the finger recess 52 flares the withdrawal end 48 of the tampon 42. Flaring tampons is a common practice of users to help prevent leaks and/or to aid insertion. However, the tampon 42 of the present invention has sufficient column strength to be inserted digitally without being flared. Thus, a non-flaring user need not change her habits.

Absorbent tampons are usually substantially cylindrical masses of compressed absorbent material having a central axis and a radius that defines the outer circumferential surface of the tampon, Tampons are often formed by first obtaining a shaped mass of absorbent material called a tampon blank. This blank can be in the form of a roll of sheet-like material, a segment of a continuous absorbent material, a mass of randomly or substantially uniformly oriented absorbent material, an individually prepared or cast mass of absorbent material, and the like.

The tampon blank is relatively uncompressed and has a relatively low density. The overwrap substantially encloses the tampon blank, and thus, encloses a majority of the outer surface of the finished tampon. The blank may then be compressed to form a product having overall dimensions less than those of the blank prior to use. Preferably, the tampon blank has a generally uniform fiber density along its length. This can provide sufficient fiber availability to provide a compressed fiber density along the tampon length, resulting in appropriate column strength for the tampon. The compressed tampons may have a generally uniform density throughout the tampon, or they may have regions of differing density as described in the commonly assigned patents to Friese et al., U.S. Pat. No. 6,310,296, and Leutwyler et al., U.S. Pat. No. 5,911,712, the disclosures of which are herein incorporated by reference.

The overwrap can ease the insertion of the tampon into the body cavity and can reduce the possibility of fibers being separated from the tampon. Those of ordinary skill in the art are familiar with materials that are useful in forming overwraps. Overwrap materials may be selected from an outer layer of fibers that are fused together (such as by thermobonding), a nonwoven fabric, an apertured film, or the like.

The materials that may be used in the tampon include fibers, foams, and or other discrete materials. The materials may be polymeric or cellulosic. A representative, non-limiting list of useful materials, includes fibrous materials, such as cellulose, polyester, polyvinyl alcohol, polyolefin, polyamine, polyamide, polyacrylonitrile, and the like. A representative, non-limiting list of useful cellulosic fibers includes natural fibers such as cotton, wood pulp, jute, hemp, sphagnum, and the like; and processed materials including cellulose derivatives such as regenerated cellulose (including rayon and lyocell), cellulose nitrate, carboxymethyl cellulose, and the like.

Compressed tampons rebound slightly after moderate mechanical compression toward their original dimensions. Therefore, tampon blanks are generally over-compressed to allow them to rebound slightly to the desired density for use. Over-compression mechanically constricts expansion to prevent the tampon from expanding without added liquid.

During use, the tampons of the present invention absorb moisture and liquids and radially expand. These tampons expand primarily in a direction perpendicular to the central axis of the tampon. Preferably, the tampons expand in at least one direction perpendicular to the central axis, more preferably, at least two directions. Most preferably, the tampons expand substantially uniformly in all directions perpendicular to the central axis.

Tampons are generally categorized in two classes: applicator tampons and digital tampons, and a certain amount of dimensional stability is useful for each type of tampon. Applicator tampons use a relatively rigid device to contain and protect the tampon prior to use. To insert the tampon into a body cavity, the applicator is partially inserted into the body cavity, and the tampon can be expelled therefrom. In contrast, digital tampons do not have an applicator to help guide them into the body cavity and require sufficient column strength to allow insertion without using an applicator. This strength can be determined by securing one end of the tampon to the fixed plate of an Instron Universal Testing Machine, available from Instron Corporation, Canton, Mass., USA. The moveable plate is brought to contact the opposite end of the tampon and is then set to compress the tampon at a rate of about 5 cm/minute. The force exerted on the tampon is measured continuously, and the point at which this force begins to fall instead of rise is the point at which the tampon buckles. The maximum force achieved is the tampon stability. Preferably, digital tampons of the present invention have a significant stability, at least about 10 Newtons (N). More preferably, the digital tampons have a stability of at least about 15 N, 20 N or 25 N, and most preferably, they have a stability of about 30 N to about 85 N. Tampons with a stability that is too low do not have sufficient dimensional stability to maintain their basic structure during insertion as a digital tampon; tampons with a stability which is too high can be perceived as being too stiff or hard to be comfortably inserted as a digital tampon.

As previously seen in discussion of the prior art (e.g., Friese, U.S. Pat. No. 4,816,100, FIGS. 3 and 4), the winding mandrel used to form the tampon blank 24 is a symmetrical unit, i.e., the winding mandrel has two fingers, symmetrically located about the central axis of the winding mandrel. Thus, after the absorbent web and overwrap material are rolled, the voids (shown as 26a and 26b of the FIG. 3 in the present case) are also symmetrical in size and location.

A process for producing a tampon according to the present invention is a modification of that described generally in Friese, U.S. Pat. No. 4,816,100, Friese et al., U.S. Pat. No. 6,310,296, and Leutwyler et al., U.S. Pat. No. 5,911,712, the disclosures of which are herein incorporated by reference. An optional departure from Friese '100 is a modification of the winding mandrel. Significant departures from Friese et al. '296, and Leutwyler et al. '712 are modifications in the press section. These departures and additional features are discussed in detail, below.

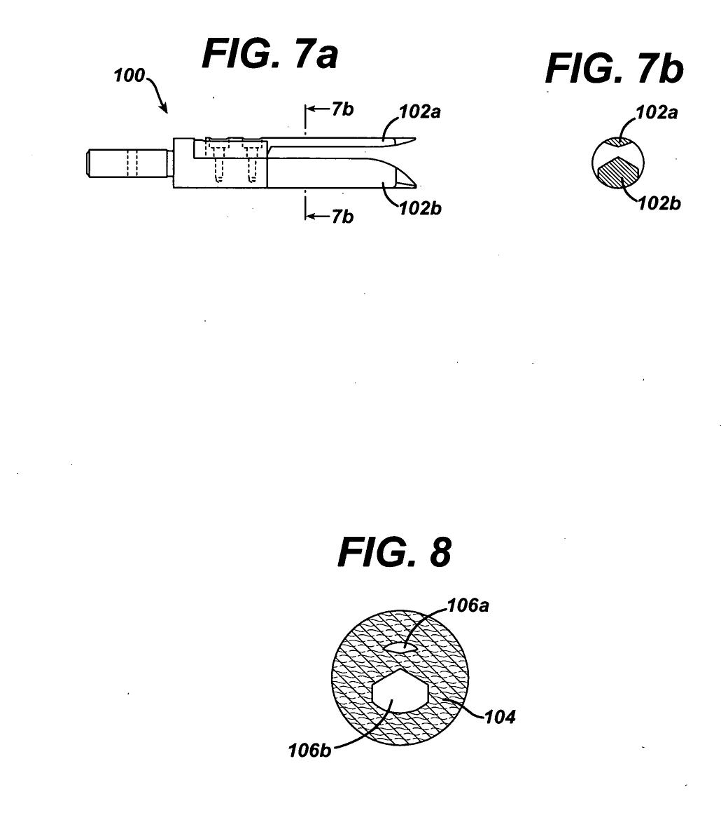

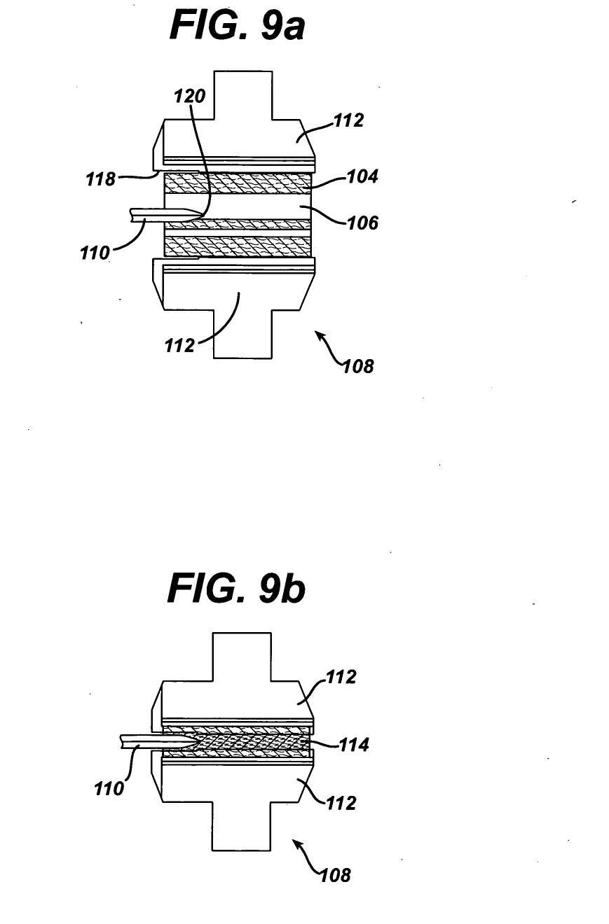

In manufacturing a tampon according to the present invention, the absorbent web is rolled up about a winding mandrel to form a loose, cylindrical tampon blank. The winding mandrel can be similar to that disclosed in Friese, U.S. Pat. No. 4,816,100 (FIGS. 3 and 4), and form the two similar voids. Alternatively, the winding mandrel can be asymmetrical. An example of an asymmetrical winding mandrel 100 useful in the present invention has two dissimilar fingers 102a and b. FIG. 7 illustrates one example; one finger may be substantially larger than the other. Additionally, the fingers may not be symmetrically located about the longitudinal axis of the winding mandrel. When this winding mandrel 100 is used to roll the absorbent web to form the rolled tampon blank 104, it produces two formed voids, 106 a and b, one of which (106b) includes the longitudinal axis of the tampon blank. This is shown in FIG. 8. These voids allow formation of the finger recess without significant displacement of fibers along the longitudinal axis.

After the tampon blank 104 has been formed, it is placed into a press 108 similar to that shown in Leutwyler et al. '712. However, a forming mandrel 110 is inserted into one end of a tampon blank while the tampon blank is positioned in the press. While the forming mandrel 110 can simply be inserted into the tampon blank and form its own space by moving the fibers of the absorbent structure, it is preferred that it be inserted into a void formed by the winding mandrel. If an asymmetrical winding mandrel 100 is used, it the forming mandrel 110 can fit into the larger void 106b of the tampon blank 104. This forming mandrel 110 keeps fibers out of the space for the finger recess 52. Again, this forming mandrel 110 eliminates the need to move fibers along the longitudinal axis. The jaws 112 of the press compress the blank while the forming mandrel 110 remains in the structure that becomes a compressed tampon 114.

In one embodiment, the forming mandrel 110 is a formed stainless steel rod. When using a stainless steel rod, it is preferred that the compression jaws 112 of the press 108 that compress the blank 104 around the rod be partially removed (e.g., at 118). In other words, the press jaws extend less toward the central longitudinal press axis in a portion corresponding to the forming mandrel. The shortening of the press jaws allows for compensation of the volume that the rod is occupying within the tampon blank 104. Of course, one of ordinary skill in art will recognize that some arrangements of jaws and forming mandrel may not require this shortening.

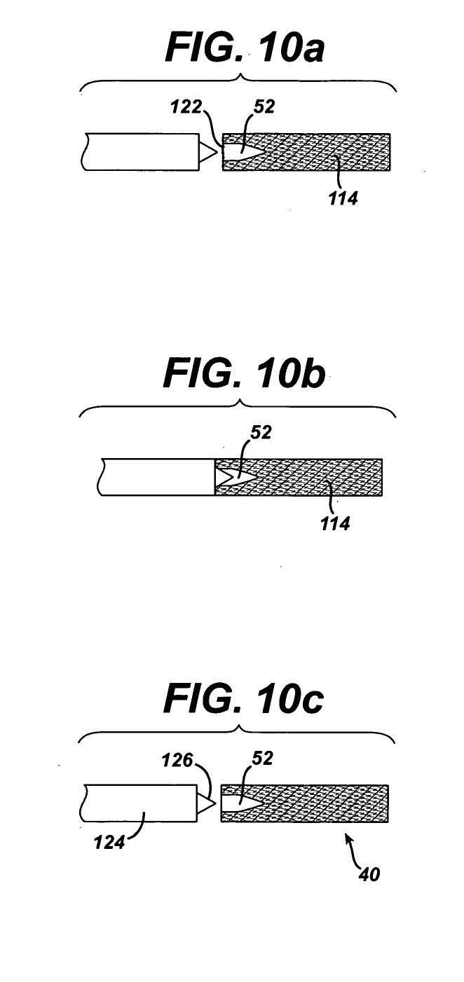

The outer diameter of the tampon remains relatively constant from the insertion end to withdrawal end. Additionally, the density along the longitudinal axis of the tampon (the central core) remains relatively constant from the insertion end to the innermost portion of the finger recess 52 (as shown in FIGS. 4 and 10).

The forming mandrel can also be made from other materials. For example, a resilient foam structure may be inserted into the blank. Once the blank is compressed, the foam may be removed. It has been found that if a pliable piece of material is used as a forming mandrel, the void formed with the withdrawal end may be star-shaped, that is, an inverse of the ribs formed by the press jaws.

Additionally, the forming mandrel may be of any shape. In one embodiment, the forming mandrel is cylindrical, with a tapered leading end 120, other embodiments of the forming mandrel 110 are also possible. For example, it may have a multi-pointed star cross-section, polygonal, and the like. Preferably, the dimensions of the forming mandrel are in accordance with the dimensions of the tampon. For example, for most vaginal tampons, the mandrel may be between about 2 mm and about 8 mm in diameter, preferably, between about 3 mm and about 6 mm. This typically provides clearance between the forming mandrel and press jaws of at least about 0.2 mm, preferably between about 0.2 mm and about 1.5 mm, and most preferably between about 0.4 mm and about 0.6 mm.

The forming mandrel 110 may have additional features to assist in forming a stable finger recess 52 in a stable, compressed tampon 112. For example, means to heat the mandrel may be used to help to “heat-set” the fibers of the compressed structure. In addition, the forming mandrel may have coatings or other treatments to improve its working life, to reduce process friction, to impart desired characteristics to the finger recess, and the like.

Without sufficient design consideration, the size of the finger recess 52 could significantly weaken the product. Thus, the radial compression of the tampon 42 at the withdrawal end 48, including a substantial amount of fibers, provides a structurally stable base. Thus, the tampon 42 can be inserted digitally, with or without being flared (finger protection). The user need not change her habits, whether or not she flares the product. It also provides significant column strength, as discussed above. The column strength of the present invention can be measured for the tampon overall, for the withdrawal end portion containing the finger recess, and/or for that portion of the tampon that excludes the finger recess. These variations can be determined by measuring sections of the tampon.

Notwithstanding the presence of the substantial finger recess, the column strength of the present invention can compare favorably with the commercially available o.b.® Normal Absorbency Tampon.

Turning to FIG. 10, once the compressed tampon 114 is removed from the press 108 for further processing, the finger recess 52 may be partially obstructed by fibers 122 extending from the edges of the compressed tampon 114 as well as the withdrawal string (not shown).

To address this, a transfer rod 124 having a tip 126 may be used to transfer the finished tampon 42. The tip 126 is inserted into the finger recess 52 to align the string to the outer perimeter of the compressed tampon 114 and to suppress any loose fibers 122 remaining about or in the void from the pressing operation (removal of the forming mandrel 110). The tip 126 may be a smooth continuation of the transfer rod 124, or there may be a bearing surface 128 adjacent the base of the tip 126.

The transfer rod 124 may be used at one of several locations in the manufacturing process, but one location it is particularly useful is in moving the compressed tampon into the dome-former (not shown) to produce the finished tampon 42. The transfer rod 124 and tip 126 can take on any useful form, depending upon the desired outcome. For example, the tip can be conical, elliptical, cylindrical, and the like. The tip should be appropriately dimensioned in connection with the finger recess to penetrate into the finger recess no more than necessary. Thus, the tip 126 should have a length less than or equal to the depth of the finger recess. For example, the tip can have a length of less than about 15 mm, preferably less than about 10 mm, or even less than about 5 mm. The tip may be conical as shown in FIG. 10. The included angle of the cone can be between about 15° and about 120°, depending upon other design factors recognized by one of ordinary skill in the art. Like the forming mandrel, the transfer rod 124 and especially the tip 126 may be heated or unheated.

The specification and embodiments above are presented to aid in the complete and non-limiting understanding of the invention disclosed herein. Since many variations and embodiments of the invention can be made without departing from its spirit and scope, the invention resides in the claims hereinafter appended.

We claim:

1. A tampon comprising: a fibrous absorbent body having:
a) an insertion end, a withdrawal end, and a longitudinal axis;
b) a finger recess having a depth of at least about 5 mm formed into the withdrawal end;
c) a column strength of at least about 10 Newtons (N);
d) a generally uniform fiber distribution along the length of the tampon.

2. The tampon of claim 1, further comprising a cover forming an outer surface of the fibrous absorbent body.

3. The tampon of claim 1, further comprising a withdrawal sting extending from the withdrawal end of the fibrous absorbent body.

4. The tampon of claim 1, wherein the finger recess is sized to accommodate a user's finger.

5. The tampon of claim 1, wherein the finger recess has a diameter, and a ratio of depth of finger recess to diameter of finger recess is at least about 1:1.

6. The tampon of claim 1, wherein the finger recess is bounded by an annulus of relatively high fiber density, which annulus is in turn bounded by an outer region of relatively low fiber density.

7. The tampon of claim 1, wherein the fibrous absorbent structure comprises a core of relatively high fiber density substantially surrounding the longitudinal axis, from which a plurality of ribs of relatively low fiber density extend radially.

8. The tampon of claim 7, wherein the finger recess is surrounded by the core at the withdrawal end.

9. The tampon of claim 8, wherein the core has a greater diameter proximate the withdrawal end than proximate the introduction end.

10. The tampon of claim 7, wherein ribs are generally separated from adjacent ribs where they extend from the core by an open channel.

11. The tampon of claim 7, wherein the ribs are spirally shaped along the tampon.

12. A method of forming tampon, the method comprising the steps of:
a) winding an absorbent fibrous web around a winding mandrel;
b) transferring the blank into a press, the press having a plurality of press jaws;
c) inserting a forming mandrel into one end of a tampon blank while the tampon blank is positioned in the press;
d) moving the jaws toward a central longitudinal press axis to compress the tampon blank and to form a compressed tampon having a finger recess formed into the one end of the tampon; and
e) ejecting the compressed tampon from the press.

13. The method of claim 12, wherein the winding mandrel has a first finger and a second finger to form a tampon blank having a first formed void corresponding to the first finger and a second void.

14. The method of claim 13, wherein the step of inserting a forming mandrel into one end of a tampon blank comprises inserting the forming mandrel into one of the first and second voids.

15. The method of claim 13, wherein the first finger is larger than the second, and the first void is larger than the second void.

16. The method of claim 15, wherein the step of inserting a forming mandrel into one end of a tampon blank comprises inserting the forming mandrel into the first void.

17. The method of claim 12, further comprising the step of pressing into the finger recess to transfer the compressed tampon during manufacture.

18. The method of claim 12, wherein the compressed tampon has an outer diameter that is substantially constant.

19. The method of claim 12, wherein the finger recess has a depth of at least about 5 mm.

20. The method of claim 12, wherein the press jaws extend less toward the central longitudinal press axis in a portion corresponding to the forming mandrel.

21. An apparatus for manufacturing an absorbent tampon having an insertion end, a withdrawal end, and a longitudinal axis, the apparatus comprising:
a) a plurality of press jaws, radially moveable toward a central longitudinal press axis corresponding to the longitudinal axis of the tampon;
b) a forming mandrel insertable into the press along the central longitudinal press axis in a location corresponding to the withdrawal end of the tampon;
wherein the press jaws are radially moveable while the forming mandrel is inserted into an end of the press corresponding to the withdrawal end of the tampon.

22. The apparatus of claim 21, wherein the press jaws extend less toward the central longitudinal press axis in a portion corresponding to the forming mandrel.

23. The apparatus of claim 21, wherein the forming mandrel has a circular cross-section;

24. The apparatus of claim 21, wherein the forming mandrel has a multi-pointed star cross-section.

25. The apparatus of claim 21, wherein the distal end of the forming mandrel extending into the press is tapered.

26. The apparatus of claim 21, further comprising a transfer rod:
a) capable of bearing on the withdrawal end of the tampon; and
b) comprising a tip that is insertable into the finger recess.

27. The apparatus of claim 26, wherein the tip is tapered.

28. A method of using an absorbent tampon, the method comprising the steps of:
a) removing from a package, a tampon having a withdrawal end and a finger recess located in the withdrawal end;
b) inserting a finger into the finger recess securely enough to maintain control of the tampon during insertion into a body cavity; and
c) inserting the tampon into the body cavity.
